(19) United States
(12) Patent Application Publication
(10) Pub. No.: US 2012/0083472 A1
(43) Pub. Date: Apr. 5, 2012

(54) PHARMACEUTICAL COMPOSITION FOR PROMOTING THE HEALING OF WOUNDS AND CONTAINING LYSOPHOSPHATIDIC ACID AND AN ADENYLYL CYCLASE INHIBITOR AS ACTIVE INGREDIENTS

(75) Inventors: Sang Chul Park, Gyeonggi-do (KR); Eui Ju Yeo, Gyeonggi-do (KR); Ji Heon Rhim, Seoul (KR)

(73) Assignee: SNU R&DB FOUNDATION, Seoul (KR)

(21) Appl. No.: 13/322,874

(22) PCT Filed: Apr. 26, 2010
(86) PCT No.: PCT/KR10/02596

§ 371 Date: Nov. 28, 2011

(30) Foreign Application Priority Data
Apr. 28, 2009 (KR) 10-2009-0037299

(51) Int. Cl. A61K 31/661; A61P 17/02

(52) U.S. Cl. 514/120

(57) ABSTRACT

The present invention relates to a pharmaceutical composition for promoting the healing of wounds, containing lysophosphatidic acid and an adenylyl cyclase inhibitor as active ingredients, and more particularly, to a pharmaceutical composition which contains an adenylyl cyclase inhibitor and which promotes the healing of wounds in senescent cells or aged persons having physiological characteristics different than those of young cells or young persons. In addition, the present invention relates to a method for promoting the healing of wounds involving treating a wounded person with an effective dose of the lysophosphatidic acid and adenylyl cyclase inhibitor.

### TECHNICAL FIELD

The present invention relates to a pharmaceutical composition for promoting wound healing comprising lysophosphatidic acid and adenylyl cyclase inhibitor as active ingredients, and more specifically, to a pharmaceutical composition for promoting wound healing of aged cells and aged objects which have different physiological properties from young cells and young objects, comprising lysophosphatidic acid and adenylyl cyclase inhibitor as active ingredients and a method for promoting wound healing.

### BACKGROUND ART

When a tissue is damaged, our body starts a response of healing the damage. Wound healing is comprised of complex biological procedure carried with extracellular matrix, blood cells, mesenchymal cells and intermediates. There are four phases in healing process: inflammatory phase, epithelialization phase, proliferative phase and maturation phase. And it may take a considerably long period depending on the degree of damage. Meanwhile, the period for healing wound longer, the probability of creating a scar higher and there is a apprehension of secondary inflammation. There is a need for development of material for prevention of a scar, prevention of secondary inflammation and promotion of rapid wound healing.

Fibroblasts are the major source of extracellular connective tissue matrix. In response to injury, fibroblasts rapidly proliferate and migrate from connective tissue into the injured site and participate in wound healing. Thus, by modulation of fibroblast proliferation and migration, one might be able to regulate wound healing response.

Meanwhile, it was previously shown that wound healing can be promoted in vivo by application of tyrosine kinase agonists such as PGF or EGF (Jahovic et al., 2004; Wang et al., 1996). The naturally occurring phopholipid growth factor, lysophosphatidic acid (LPA), was also reported to promote in vitro wounding healing (Balazs et al., 2001). In addition, the inventors compared the LPA receptor system with the receptor tyrosine kinase systems such as PDGF and EGF in senescent cells. The inventors observed that the receptor tyrosine kinase systems which are a receptor of PDGF (Yeo et al., 2002) and EGF (Cho et al., 2003; Park et al., 2000) are entirely reduced in senescent cells, and thus even though external stimulation is given, the stimulation is not delivered to the senescent cells due to the almost complete blockage of signal transduction and cell proliferation, however, LPA receptor is partially reduced to be some degree present, resulting in occurrence of signal transduction and cell proliferation by LPA (Jang et al., 2003).

LPA is known to be a material that induces a number of physiological responses, including platelet aggregation, smooth muscle contraction, cell shape change, chemotaxis, proliferation, and differentiation (Fukushima and Chun, 2001; Goetzl and An, 1998; Moolenaar, 2000; Piazza et al., 1995). LPA activates specific G-protein-coupled receptors (An et al., 1998; Bandoh et al., 1999; Erickson et al., 2000; Noguchi et al., 2003), thereby triggering an increase in cytoplasmic calcium, stimulation of phospholipases, activation of PI3K or of the Ras-Raf-MAP kinase cascade, and inhibition of adenylyl cyclase (Contos et al., 2000; Fukushima and Chun, 2001; Fukushima et al., 2001; Takuwa et al., 2002).

Interestingly, LPA was found to induce cAMP accumulation (Jang et al., 2006a; Jang et al., 2003) and subsequent PKA activation (Jang et al., 2006b) in senescent cells, while reducing cAMP levels in young cells. Increase of cAMP level has an ability of inhibiting cell proliferation and migration (Liu et al., 1986). The cAMP analogue dibutyryl cAMP (dbcAMP) is often used to inhibit cell proliferation by being mediated (by activation) by PKA (Liu et al., 1986). cAMP-mediated inhibition of cell proliferation may be independent of nuclear translocation of the PKA catalytic sub-units (Seternes et al., 1999). Activated PKA might cause Raf inhibition via ser/thr phosphorylation followed by inhibition of ERK activity (Chuang et al., 1994; Mischak et al., 1996; Wu et al., 1993). In previous study, the inventors showed that adenylyl cyclase inhibitor SQ22536(ACI)(Fabbri et al., 1991) regulates phosphorylation of AMPKα, thereby inhibiting catalytic activity of AMPKα and p53 to induce cell proliferation in senescent cells (Rhim et al., 2008).

It is well known that the degree of wound healing is different depending on age in aged cells, aged animals, and elderly people and wound healing is not well done with aging (Agren et al., 1999; Hardman and Ashcroft, 2008; Marcus et al., 2000; Spindler et al., 1995 Jeong et al., 2008). Senescent cells are more present in the skin of aged persons than that of younger persons, and have a reduction in the number of cellular replications needed to form granular tissues, and thus show a reduced rate of wound healing (Pitterman, 2007). While the preponderance of existing evidence supports the view that cellular aging impairs wound healing, a precise understanding of the underlying mechanisms remains elusive.

### DISCLOSURE

### Technical Problem

Therefore, the inventors have focused on processes of cellular senescene and related factors in slowed or impaired wound healing in the elderly. As a result, the inventors found the fact that wound healing of aged animal can be regulated by treating cAMP level increased by treating with lysophosphatidic acid (LPA) with adenylyl cyclase inhibitor (ACI) to regulate cellular cAMP level, thereby completing the present invention.

### Technical Solution

It is an object of the present invention to provide a composition for promoting wound healing of aged cell and old subject which have different physiological properties from young cell and young subject, comprising lysophosphatidic acid and adenylyl cyclase inhibitor as active ingredients.

It is another object of the present invention to provide a method for promoting wound healing, containing the step of treating a subject having wound with effective dose of lysophosphatidic acid and adenylyl cyclase inhibitor.

Other objects and advantages of the present invention are disclosed by the appended claims and the following embodiments including figures.

In addition, a number of cited references are referred through the specification and the citations are expressed. The disclosure of cited references and patents is incorporated as a whole into the present invention as a reference to explain more clearly the level of art that the present invention pertains to and the content of the present invention.

### Advantageous Effect

The present invention relates to a pharmaceutical composition for promoting wound healing comprising lysophosphatidic acid and adenylyl cyclase, more specifically a pharmaceutical composition for promoting wound healing of aged cells and old subjects which have different physiological properties from young cells and young subjects, comprising lysophosphatidic acid and adenylyl cyclase.

In addition, the present invention relates to a method for promoting wound healing, containing the step of treating a subject with effective dose of lysophosphatidic acid and adenylyl cyclase inhibitor.

The composition according to the present invention has an excellent effect of wound healing of aged cells and old individuals.

### DESCRIPTION OF DRAWINGS

The above and other objects, features and advantages of the present invention will become apparent from the following description of preferred embodiments given in conjunction with the accompanying drawings, in which:

FIG. 1 shows the wounding surgery of Rat Back. Rats were anesthetized with intraperitoneal injection of ketamine and xylazine. The back was shaved (A) and four full-thickness circular skin wounds of 1.0 cm diameter were cut (B). A teflon wound chamber was glued into the edge of the skin and then sewn using a surgical steel wire (C).

FIG. 2 shows reduction of cellular cAMP by ACI in young and senescent cells. Sub-cultured young (PD 20) and senescent (PD 65) fibroblasts were treated with various concentrations of ACI for 1 hr. The level of cAMP in the acid extracts was measured by using a cAMP binding assay. The results shown represent the means±standard deviation of a representative experiment.

FIG. 3 shows an effect of a cAMP analog and an ACI on LPA-induced cell growth in young and senescent cells. Sub-cultured young (PD 25, A) and senescent (PD 73, B) fibroblasts were serum-starved for 2 days and treated with LPA, 1 mM of dbcAMP (+dbcAMP), or 300 μM of ACI SQ22536 (+ACI) for 4 days. The number of cells was counted. The results shown represent the means±standard deviation of a representative experiment.

FIG. 4 shows effects of a cAMP analog and an ACI on thymidine incorporation in young and senescent cells. Sub-cultured young (PD 25) and senescent (PD 73) cells (A), skin fibroblasts from young and elderly donors (B), HUVEC (C) and skin keratinocytes (D) were serum-starved for 2 days and treated with 1 mM of dibutyryl-cAMP (dbcAMP), or 300 □μM of ACI (SQ22536) for 2 hrs. Cells were then treated with 30 μM of LPA in the presence of 0.5 μCi/ml of [³H]-radioactive isotope. The radioactivity incorporated into the DNA was counted and fold increase of radioactive isotope incorporation was calculated and plotted. The results shown represent the means±standard deviation of a representative experiment.

FIG. 5 shows effects of a cAMP analog and an ACI on cell growth in young and senescent cells. Sub-cultured young (PD 25) and senescent (PD 73) cells (A), fibroblasts from young and elderly donors (B), HUVEC (C) and skin keratinocytes (D) were serum-starved for 2 days and treated with LPA, and then treated with 1 mM of dibutyryl-cAMP, or 300 μM of ACI (SQ22536) for 4 days. The number of cells was counted. The results shown represent the means±standard deviation of a representative experiment.

FIG. 6 shows effects of a cAMP analog and an ACI on LPA-induced cell migration. Sub-cultured young (A, C) and senescent cells (B, D) were serum-starved for 2 days and treated with ACI or dbcAMP (1 mM) for 2 hr at 37° C. An area was denuded by a yellow tip and the cells were then treated with LPA (30 μM) and incubated at 37° C. Photographs were taken at 6 hrs, 12 hrs, 18 hrs, 24 hrs, 30 hrs, 36 hrs, 42 hrs, 60 hrs, and 72 hrs, and the number of cells migrated into the acellular area was counted and the average values were plotted in A and B. The data at 72 hrs were replotted in a bar graph to show the significant changes induced by treating cells with either LPA or ACI, or both (C and D). The results shown represent the means±standard deviation of a representative experiment. Cell migration from each group (p<0.001) was compared to the control value the migration was read as having a significance.

FIG. 7 shows effects of LPA and an ACI on wound healing. Young and old rats were anesthetized and wounded as described in the legend of FIG. 1. 200 μl aliquots of the solutions containing LPA (30 μM), ACI (300 μM), and the mixture of LPA and ACI dissolved in physiological saline were applied onto wound sites using a sterile glass filter in each wound chamber once per two days. After 4 days, the wounds were photographed.

FIG. 8 Shows epithelialization of wounds visualized by H & E staining. Young and old rats were anesthetized and wounded as described in the legend of FIG. 1. The wounds were treated with LPA (30 μM), ACI (300 μM), or the mixture of LPA and ACI as described in the legend of FIG. 1c. On postoperative days 4, animals were anesthetized and killed as described in “Materials and Methods”. Approximately 5-mm-thick section across the dorsoventral diameter of the wound was cut and fixed and embedded in paraffin. Serial sections were cut and processed for hematoxylin-eosin (H & E) staining. H & E stained epithelial layer was visualized on the top of each section.

FIG. 9 shows wound healing visualized by PCNA staining. Young and aged rats were anesthetized and wounded as described in the legend of FIG. 1. The wounds were treated with LPA (30 μM), ACI (300 μM), or the mixture of LPA and ACI as described in the legend of FIG. 1c. On postoperative days 2, 4, and 7, animals were anesthetized and killed as described in “Materials and Methods”. An about 5-mm-thick section across the dorsoventral diameter of the wound was cut and fixed and embedded in paraffin. Serial sections were cut and processed for immunohistochemistry using PCNA as described in “Materials and Methods”. The presence of PCNA was visualized by using avidin-biotin peroxidase complex method and the control was counterstained with hematoxylin. The photographs from postoperative days 4 are shown in A. The number of PCNA positive cell was counted from each photograph and plotted in B (young rats) and C (aged rats).

### BEST MODE

The invention relates to a composition for promoting wound healing in aged cell and aged object, comprising lysophosphatidic acid and adenylyl cyclase inhibitor.

Lysophosphatidic acid (LPA) is an important mitogen agonist which induces identical signal transduction in relation to intracellular Ca₂+ transport, actin polymerization and production of phosphatidic acid in human diploidy fibroblasts and acts as extracellular messenger through guanine nucleotide binding protein (G-protein). LPA is also known as the material having various biological effects on cell morphology, chemotaxis and differentiation mediated by LPA receptor (Moolenaar, 2000; Moolenaar et al., 1997). LPA receptor is exemplified by such isotypes as LPA1, LPA2 and LPA3 and these isotypes are bound to Giα which is sensitive to pertussis toxin to inhibit adenylyl cyclase activity (An et al., 1998), resulting in the decrease of cAMP (Taussig et al., 1993). Only, as previously described, LPA receptor is known to increase LPArk cAMP in senescent cell (Jang et al., 2006; Jang et al., 2003).

The composition of the present invention can be used to heal wound, specifically wound of senescent cell or skin wound of old individuals, wherein the depth of the wound is shallow or deep and the wound includes damage of corium and epidermis.

In a preferred embodiment of the present invention, when ACI and LPA are used in senescent cells and wounded old individuals, they not only increase cell proliferation but also rapidly migrate cells to wounded sites and promote wound healing differently from young cells or young individuals. In addition, it was found that co-treatment of LPA and ACI brings greater effect on the migration of cells than single treatment of LPA or ACI. This phenomenon is not consistent with that in young cells. In addition, it was found that upon co-treatment of LPA and ACI in wounded sites of old individuals, the wounded sites are more efficiently healed, compared to single treatment of LPA or ACT.

For the composition for promoting wound healing for senescent cells of the present invention, the treatment with LPA and ACI includes simultaneous treatment and sequential treatment regardless of order.

The target subject of senescent cells and old individuals in the present invention is animal, preferably mammals, most preferably human.

In addition, the term “old” in the present invention means more than age which is determined by extracting average life×0.1 from average life. For example, for a person whose average life is 65 years, his age is calculated as follows: 65−(65×0.1)=58.5. That is, for a person aged 59 years or more, he may be regarded as old individual. Herein, a person aged 60 years or more is regarded as old individual.

In addition, the said adenylyl cyclase inhibitor (ACI) is selected from 2′,5′-dideoxyadenosine, cis-N-(2-phenylcyclopentyl)azacyclotridec-1-en-2-amine (MDL12,330A hydrochloride), and 9-(tetrahydro-2′-furyl)adenine (SQ22536), and more preferably 9-(tetrahydro-2′ furyl)adenine, but not always limited thereto.

Herein, the term “senescene” has the same meaning as “aging”

The term “wound” used herein means cut, cleaved, broken, burn or externally damaged tissue, or damage resulting from disorder or disease inducing such damage.

Also, the term “healing” herein means that the period from the time point at which wound occurs to the time point at which wound entirely shrinkages is promoted or accelerated.

Herein, the term “tissue” means cell mass in an object in which cells constitutes a group to form a specific function and its examples include bone, skin, connective tissue and nerve, but not always limited thereto.

The terms “treatment” and “treating” mean healing method. In addition, the “therapeutically effective amount” means a minimum amount that is needed to give a subject therapeutically benefits. For example, “therapeutically effective amount” for an human suffering from wound means an amount that induces, promotes, accelerates or improves pathological symptoms, progress of healing, and physiological state or alleviates healing resistance delaying healing.

The pharmaceutical composition of the present invention may comprises pharmaceutically acceptable carrier, excipient or stabilizer that are not toxic to human who is exposed to administered amount and concentration. Physiologically acceptable carrier is often pH buffer solution. Examples of physiologically acceptable carrier include buffer such as phosphate, citrate and other organic acid; hydrophilic polymer; amino acid such as glycine, glutamine, asparagines, arginine or lysine; monosaccharide, disaccharide, and other carbohydrate including glucose, mannose or dextrin; chelating agent, for example, EDTA; sugar alcohol, for example, mannitol or sorbitol; salt-forming counter ion, for example, sodium; and/or non-ionic surfactant, for example, polyoxyethylene sorbitan fatty acid ester, polyethylene glycol, and polyethylene/polyoxypropylene copolymer, but not always limited thereto.

The composition of the present invention can be formulated into a pharmaceutical formulation by a conventional method as known in the art.

In addition, the pharmaceutical composition of the present invention can comprise suitable pharmaceutical diluent which is known to be useful in a pharmaceutical composition. Examples of the diluent include saline, buffered saline, extrose, water, glycerol, ethanol and combination thereof, but not always limited thereto. The composition of the present invention can additionally contain lubricants, wetting agents, sweeteners, aromatics, emulsifiers, suspending agents, and preservatives, but not always limited thereto.

The composition of the present invention can be formulated by the method that can be performed easily by those in the art by using a pharmaceutically acceptable carrier and/or excipient in the form of unit dose or in multi-dose container. The formulation can be in the form of solution, suspension or emulsion in oil or water-soluble medium, extract, powder, granule, tablet or capsule. To maintain the maximum physiological activity of the active ingredient, a buffer containing proper amount of salt and pH regulator can be added. In addition, dispersing agent or a stabilizer can be additionally included so that active ingredient can act effectively.

Suitable pharmaceutically acceptable carrier and formulation are detailedly described in Remington's Pharmaceutical Science (19^{th} ed., 1995).

The composition must be suitable for administration method, and can be administered by conventional method, for example, through oral and parenteral administration pathway. For parenteral administration, the composition can be administrated through rectal, topical, intravenous, intraperitoneal, intramuscular, intraarticular, subcutaneous, nasal, inhalation, intraocular, or intradermal pathway.

The effective dose of the composition of the present invention varies depending on formulation method, administration pathway, age, weight, gender, health condition, diet, administration frequency, administration method, excretion and sensitivity, and can be generally easily determined and prescribed by an experienced doctor by considering the effectiveness in desired prevention or treatment and the dose can be determined to be administered once or several times daily.

In accordance with another embodiment of the present invention, the present invention relates to a method for promoting wound healing by treating aged cells and aged subject with efficient amount of LPA and ACI.

In accordance with preferred embodiment, the target subject for the wound healing is animal, preferably mammals, most preferably human individual.

Among the method and described composition of the present invention, overlapped content is not described to avoid complex of the specification. In addition, Unless stated otherwise, every technological and scientific terms used in this invention are understood as conventional meaning accepted by those in the art.

Hereinafter, the present invention will be detailedly illustrated by reference to examples. These examples only intend to detailedly illustrate the present invention. It is obvious to a person skilled in the art that the present invention pertains to that the spirit and scope of the present invention are not limited by these examples.

### Examples

1. Materials

The following reagents were used in the present invention: Dulbecco's modified Eagle's medium (DMEM) was used for culturing cells, and dibutyryl-cAMP, 5-bromo-2-deoxyuridine (BrdU), eosin, hematoxylin, and peroxidase (HRPO)-labeled anti-mouse (goat) secondary antibody (rat serum protein-absorbed) from Sigma, USA; fetal bovine serum, antibiotics containing penicillin and streptomycin from Gibco/BRL Life Technologies, Inc, USA; common ACI SQ22536 from Calbiochem, San Diego, Calif., USA; cAMP assay kit, [³H]-cAMP, and [³H]-thymidine from Amersham Pharmacia Biotech, Buckinghamshire, UK; methoxyflurane (Metophane) from Smith Kline Beecham; Trypsin and Zymed picture plus kit from Zymed; anti-PCNA (NCL-PCNA) antibodies form novocastra; anti-Brd-U IgG₁, anti-PCNA monoclonal IgG, Protein block serum free, and the avidin-biotin peroxidase complex (LSAB) kit from Dako. Copenhagen, Denmark; electric diamond bar from Academy Science Korea; permount and Histoacryl from Fisher Scientific, Atlanta Ga., USA were used as materials in experiments.

2. Animals

Rats aged 6 months and 24 months which were used in experiments were purchased from Samtaco BioKorea (Seoul, Korea) and housed two per cage in a room with controlled temperature and humidity with a 12:12-h light-dark cycle. They were maintained on a standard diet with food and water ad libitum in an animal facility accredited by the Korean Association for Accreditation of Laboratory Animal Care. Animals were divided into treatment groups, each consisting of nine, and used in experiments.

3. Cell Culture

Human umbilical vein endothelial cells (HUVEC) were isolated from endothelial cells of umbilical cords as described by Whang et al (Whang et al., 2005). Foreskin human fibroblasts and keratinocytes were isolated from newborn foreskins as described by Boyce and Ham (Boyce and Ham, 1983). Patient consents were obtained from all donors and the use of tissue samples was approved by the ethics committee of the Seoul National University. The obtained and isolated fibroblasts and HUVEC were maintained in DMEM containing 10% fetal bovine serum (FBS) and antibiotics, and keratinotyes were in keratinocyte growth medium (KGM) containing antibiotics. Cells from the early stage of culture were cultured to obtain aged cells, which were used in experiments (population doubling -PD- of less than 25 vs PD 65-70 in fibroblasts, PD less than 5 vs PD 10-12 in case of HUVEC). Senescent cells were characterized by morphological changes, enhanced □β-galactosidase activity and a reduced rate of proliferation (Yeo et al., 2000). Cells were grown for 2 days to 60-70% sub-confluency in the culture medium after treatment with LPA or ACI, and then serum-starved to quiescence by incubating them with either serum free DMEM containing 0.1% BSA (fibroblasts or HUVEC) or KBM (keratinocytes) for 2 days.

4. Measurement of Cyclic AMP (cAMP)

Cells grown on a 12 well-plate were treated with various concentrations of ACI as indicated in the FIG. 2. After removing the medium, 2.5 M of perchloric acid was added into the plate. The obtained acid extract was stored at 20° C. until use, when the extract was neutralized with 4.2 M KOH and the cAMP content was determined by competitive binding with [³H]-cAMP to the cAMP binding protein, RIα□ of cAMP-dependent protein kinase (Jang and Juhnn, 2001). After the extraction, cells were lysed with 0.1 N NaOH and protein content was determined using Bradford protein assay reagent. The cAMP content was normalized versus the amount of acid-insoluble protein (expressed as the content of cAMP per protein concentration).

5. DNA Synthesis

Young and senescent cells were made so that their growth was inhibited using serum-free medium and then were pretreated with either vehicle (not treated with anything) or dbcAMP or ACI for 2 hrs and then treated with LPA in the presence of [³H]-thymidine (0.5 □mCi/ml) and then further incubated for 36 hrs. After removing the unlabeled radioactive thymidine by washing with PBS four times, DNA was precipitated with 10% TCA and lysed in 5N NaOH. Radioactivity in the neutralized lysates was counted in a liquid scintillation cocktail. The measured radioactivity as such was compared to that of control to find the synthesis degree of protein.

6. In Vitro Wound Healing Assay

LPA-induced cell migration was assessed by the ability of the cells to move into an acellular area (Shiraha et al., 2000). Cells were plated on a 10-cm plastic dish and grown to 70-80% confluence in the culture media with 10% FBS. After a 2-day quiescence in serum free media with 0.1% BSA, cells were pretreated with vehicle, ACI (300 μM), or dbcAMP (1 mM) for 2 hrs at 37° C. An area was denuded by a tip and the cells were then incubated at 37° C. without or with LPA (30 μM). Photographs were taken at 6 hrs, 12 hrs, 18 hrs, 24 hrs, 30 hrs, 36 hrs, 42 hrs, 60 hrs, and 72 hrs, and the number of cells migrating to the acellular area was counted.

7. Generation of Skin Wounds of an Animal and Preparation for Immunohistochemistry

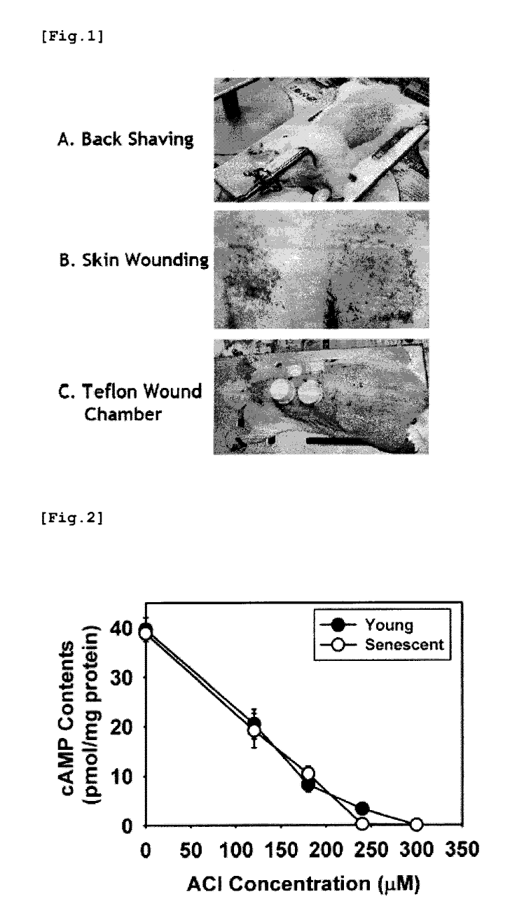

Rats were anesthetized with intraperitoneal injection of ketamine and xylazine (87/13 mg/kg). Skin wounding and its protection was performed as described by Balazs (Balazs et al., 2001). The back was shaved as shown in FIG. 1A. By the dermabrasion technique using diamond bar, four second-degreed partial thickness circular wounds of 1.0 cm. A teflon wound chamber with a 1.2 cm inner diameter centered around the wound was glued into the edge of the skin with Histoacryl and then sewn using a surgical nylon suture (Ethicon 4-0) (FIG. 1C). In order to protect wound site, upon treatment with LPA or ACI, they were sprayed using a sterile filter (GF-C Whatman), followed by treatment. This work was carried out by a single plastic surgeon for consistency and exactness of experiments. 30 μM of LPA, 300 μM of ACI, and the mixture of LPA (30 μM) and ACI (300 μM) are dissolved in physiological saline to be a 200-μl aliquot, respectively. The 200-μl aliquot, corresponding to 30 pmol/mm², was applied twice a day into the wound of the animals anesthetized with inhalation. The contralateral side received 200 μl of saline as a control.

On postoperative 2, 4 and 6 days, groups of three animals were anesthetized with ketamine and xylazine and injected with 50 mg/kg BrdU as a bolus in 500 μl saline through the jugular vein. The 45-min time interval has been shown to permit the labeling of proliferating cells in situ without much interference from cells that have migrated into the wound area and undergone DNA synthesis elsewhere. The animals were killed by intracardiac perfusion of 4% (wt/vol) paraformaldehyde in phosphate-buffered saline solution, pH 7.4. Thereafter, wound tissue was immersed in cold 10% paraformalin overnight to prepare embedded tissue, followed by section with 5 mm thickness.

8. Immunohistochemistry

Serial sections (4 □μm thick) were cut and processed for hematoxylin-eosin (H & E) and immunohistological staining. Paraffin sections on the microslides were deparaffinized in xylene and rehydrated sequentially in alcohol. Antigen retrieval was achieved by incubation with 10 mM citrate buffer (pH 6.0) in a microwave oven at 700 W for 15 min. Slides were quenched in hydrogen peroxide (3%) to block endogenous peroxidase activity and then washed in TBS buffer (0.05 M, pH 7.6). Slides were blocked in 5% blocking buffer solution for 1 hr at room temperature for nonspecific staining of protein. Slides were incubated at 4° C. overnight with primary antibody diluted in a blocking buffer. BrdU IgG₁ (1:200); PCNA mIgG (1:2,000) were used as antibodies. The avidin-biotin peroxidase complex method following the LSAB kit was used, and slides were counterstained with hematoxylin. Slides were dehydrated sequentially in ethanol, cleared with xylenes, and mounted with Permount.

9. Evaluation of Wound Healing

Through histopathological analysis, the degree of wound healing was confirmed. The degree of epidermal cell proliferation was confirmed by H&E staining. Proliferating cells were identified and calculated through the immunoperoxidase staining slide using BrdU and PCNA by Optimas image analysis program (Media cybernetics, version 6.2, Silver Spring, Md., USA). Calculated cells are percentage of relative positive stained epidermal cells in the whole length of wound.

10. Statistical Analysis

The Graph-Pad Prism (GraphPad, San Diego, Calif.) was used for statistical analysis. T-test for paired variables was used to determine whether the index was significantly different as a result of the treatment. Data are presented as the mean±standard deviation. P values of less than 0.05, 0.01, and 0.001 were marked as *, **, and ***, respectively. P values of less than 0.05 were considered to be significant.

### Results

### 1. Inhibition of AC Enhances LPA-Stimulated Proliferation of Senescent Cells

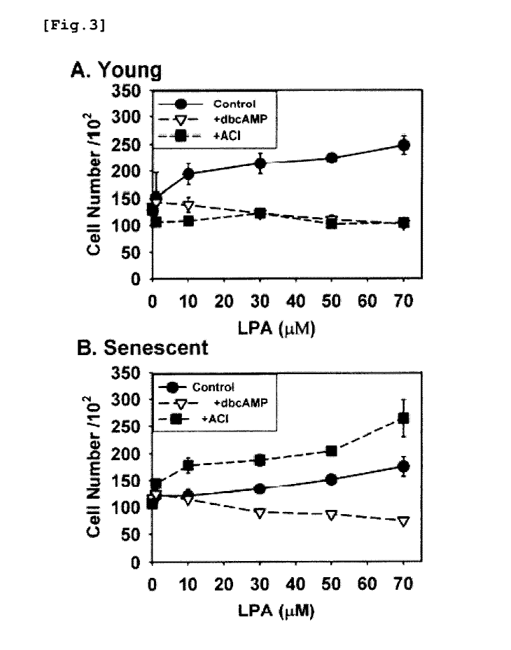

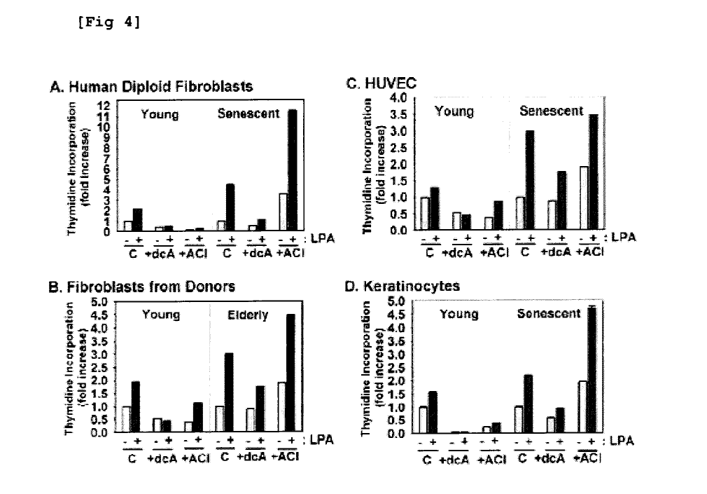

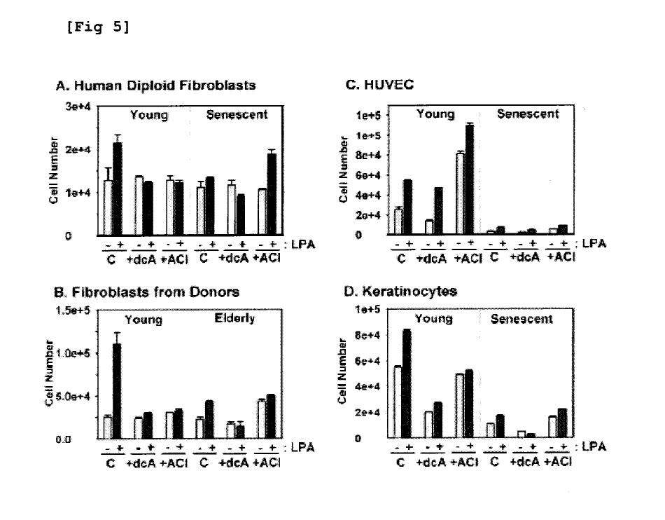

After confirming that upon treatment of fibroblast with LPA and ACI, the level of cAMP and cell proliferation ratio is modulated (upon treatment with LPA, in young cells, the concentration of cAMP is lowered, and in aged cell, that of cAMP is increased, with the treatment inducing cell proliferation in both cells. Upon treatment of aged cells with ACI, the concentration of cAMP is again lowered, and the proliferation of aged cells is more increased, compared to upon treatment with LPA. Upon co-treatment with LPA and ACI, the effect is more increased. The inventors compared the effect of a cell permeable cAMP analog (dbcAMP) and ACI (SQ22536) on LPA-induced DNA synthesis and proliferation of senescent cells such as aged fibroblasts, old donor skin fibroblasts, HUVEC, and human skin keratinocytes. The inventors found that treatment with ACI dose-dependently reduced cAMP levels, causing a complete abrogation at a concentration of 300 μM in both young and senescent cells (FIG. 2). After treating young and senescent fibroblasts with various concentrations of LPA (1-70 μM), followed by treatment with dbcAMP or ACI for 4 days, the cell number was counted (FIG. 3). As shown in FIG. 3, LPA induces proliferation of both young and senescent cells in a dose-dependent manner. Unlike young cells, senescent cells showed a significant increase in the cell number which indicates cell proliferation, following ACI treatment (FIG. 3B). LPA-induced proliferation of both young and senescent cells was decreased by treatment with dbcAMP after treatment with LPA (that is, which means that cAMP concentration is high, cell proliferation is low). The rates of DNA synthesis were also determined by measuring [³H]-thymidine incorporation into DNA (FIG. 4). Both basal and LPA-stimulated DNA synthesis decreased in senescent cells when treated with dbcAMP, but increased by treatment with ACI (FIG. 4A-Senescent cells). The effect of ACI was further tested in young and aged fibroblasts as well as HUVEC (FIG. 4C) and keratinocytes (FIG. 4D). ACI enhanced the thymidine incorporation in both basal and LPA-stimulation conditions in all these cells. The inventors also confirmed that the cell number was further enhanced by either ACI or LPA in senescent cells, although to different degrees depending on the cell type (FIG. 5). These results strongly suggest that cAMP may be a metabolic inhibitor of mitogenic stimulation and that treatment with ACI lowers cAMP content, thereby increasing DNA synthesis and cell proliferation in various senescent cells, including fibroblasts, endothelial cells and keratinocytes.

### 2. Inhibition of AC Enhances LPA-Stimulated Migration of Senescent HDFs

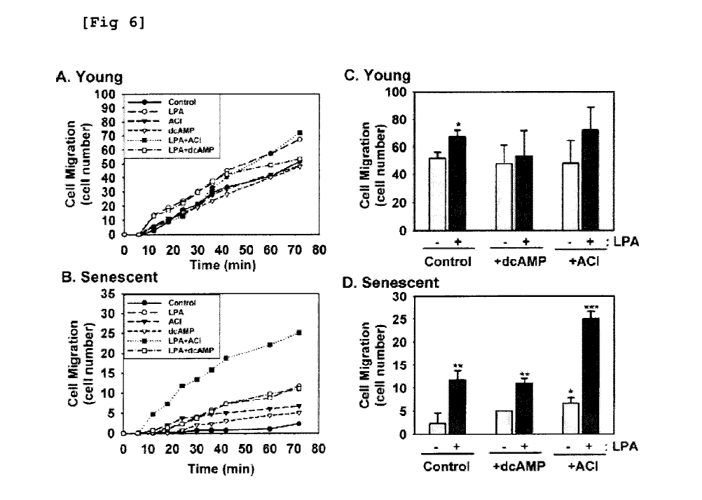

Since it was suggested that modulation of cAMP content and PKA activity might alter the rate of cell migration (Chen et al., 2005; Shiraha et al., 2002), the inventors determined the effects of dbcAMP and ACI (upon modulation of cAMP concentration) on cell proliferation, and wound healing in an animal. The cell beds of young and senescent cells were serum-starved and denuded by scraping with a sharp pipette tip. After treating the wounded cell beds with 30 μM of LPA in the presence and absence of dbcAMP and ACI, we counted the number of cells that migrated into the wound area in 72 hrs (FIGS. 6A and 6B).

The data at 72 hrs were plotted in a bar graph to show the changes induced by treating cells with LPA in addition to either dbcAMP or ACI (FIGS. 6C and 6D). As shown in FIG. 6, the young cells and young cells treated with LPA showed gradually increased migration with the lapse of time (FIG. 6C) (migration of cells is increased upon treatment with LPA, compared to upon no treatment. In contrast, the migration of senescent cells was not obvious in the absence of serum (FIG. 6B), but LPA increased senescent cell migration at 72 hrs significantly (FIG. 6D). dbcAMP did not significantly affect basal cell migration of young and senescent cells. Migration of young cells was unaltered by ACI (FIGS. 6A and 6C), but senescent cells showed an increase in cell migration by ACI alone, and treatment with both ACI and LPA enhanced cell migration more dramatically (FIGS. 6B and 6D). These data suggest that migration of senescent cells can be readily induced by LPA and further enhanced by ACI co-treatment.

### 3. AC Improves Wound Healing in Aged Fisher 344 Rats

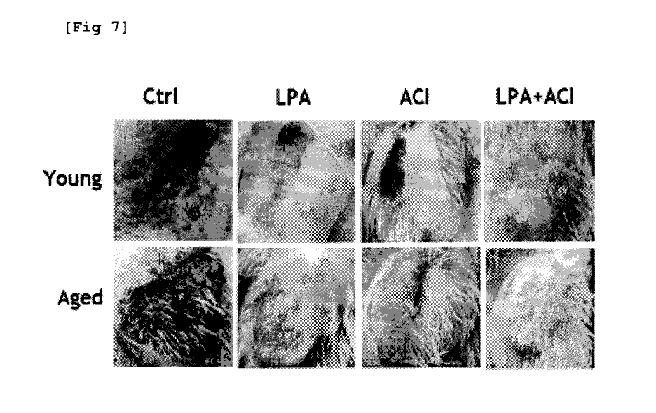

Wound healing responses to LPA and ACI were examined in both young and aged rats. LPA (30 μM), ACI (300 μM), and the mixture of LPA and ACI were applied to each wound animal once per two days. The photographs of the wounds on the 4th day show that the rates of wound healing in the young untreated control rats were faster than those in aged control rats (FIG. 7). When LPA or ACI, either alone or together, were applied to the wound animals, wound healing appeared to occur faster than the untreated controls. On postoperative day 4, slides with a section across the dorsoventral diameter of the wound were prepared as described above. Epithelialization and cell proliferation during wound healing were examined after staining with hematoxylin & eosin (H & E) and proliferating cell nuclear antigen (PCNA). After 4 days, epithelialization of young wounded rats (the H & E-stained epithelial layer visualized on the top of each section) was similar in all groups. However, epithelialization of aged wounded rats at day 4 was higher in the LPA-treated, ACI-treated, and LPA+ACI-treated groups compared to the control untreated groups (FIGS. 9A and 9B). The PCNA staining was very weak in the control aged rats, but increased significantly after treatment with LPA, ACI, and ACI+LPA in aged rats. The number of PCNA positive cells in the LPA, ACI, and LPA+ACI groups increased significantly, especially in the aged rats (FIG. 9C). These data confirm that a reduction of cAMP level by treatment of cells with ACI benefits wound healing in the aged rats.

### Discussion

Since it has been demonstrated that healing of burn wounds on the skin of animals is accelerated when they lick the wound sites, presumably by growth factors such as EGF present in the saliva, topical use of EGF-enriched artificial saliva has been considered as being helpful for wound healing (Jahovic et al., 2004). Use of PDGF, or RTK agonist, which is a potent mitogen agonist for skin fibroblasts, has also been proposed as a cure for the burn wound (Wang et al., 1996). However, it was reported that since receptors of growth factors are reduced in aged animal or aged human, such growth factors are not helpful for cell proliferation in spite of external stimulation (Yeo et al., 2002).

LPA is another agent shown to promote in vitro wound healing (Balazs et al., 2001). Through preceding study, it was confirmed that since LPA which acts by G-protein coupled receptor, is not entirely reduced in aged cells and is partially reduced (Jang et al., 2003), signal transduction or cell proliferation occurred. Therefore, it is suitable for wound healing in aged animal and aged human. The present invention demonstrates that LPA increases DNA synthesis (FIG. 4), proliferation (FIG. 5), and cell migration (FIG. 6) of senescent cells.

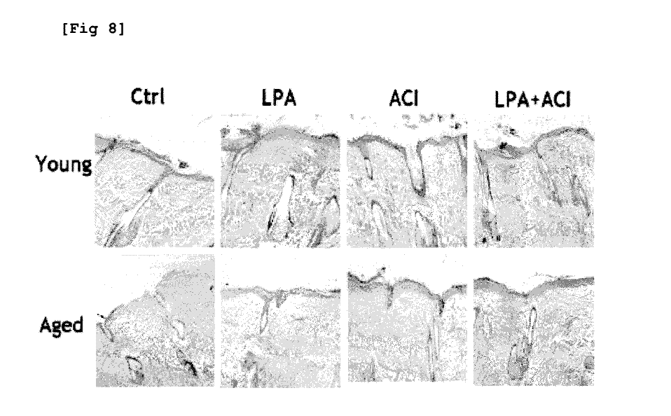

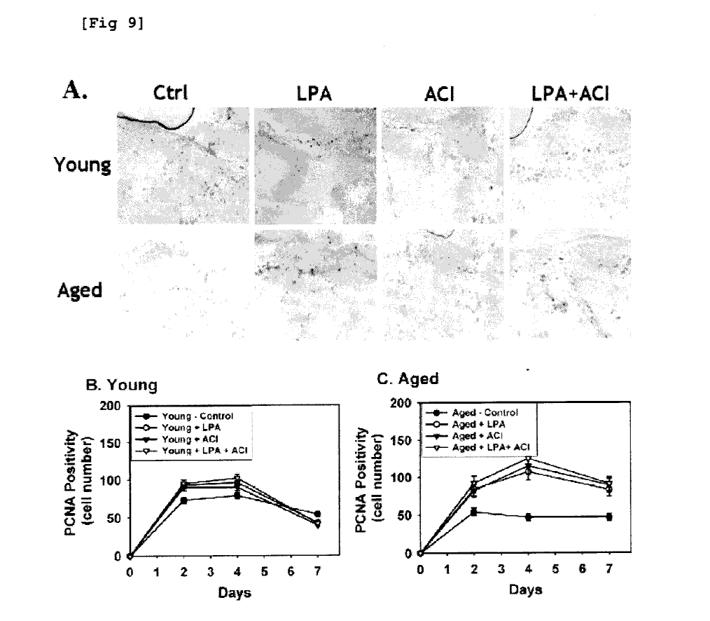

In addition, unlike EGF, LPA was effective in wound healing of aged rats (FIGS. 7, 8, and 9). LPA also increases cAMP accumulation in senescent cells compared to young cells. This increase occurs by PKC-dependent stimulation of AC isoforms 2, 4, and 6 by Giα (Jang et al., 2006a). Since it has been suggested that cAMP analogs or cAMP-producing agents increase cell proliferation, the inventors postulated that increased cAMP level would be a factor that reduces DNA synthesis or cell proliferation. Therefore, the inventors thought that senescent cell proliferation may be modulated by modulating cAMP level of senescent cell. The inventors did indeed, observe proliferation of senescent cells by treatment with a cAMP analog or dbcAMP (FIG. 3), and an enhancement of the mitogenic responsiveness of these cells by ACI treatment (Rhim et al., 2008, in press). Our findings are in accord with the observation that the TIMP-2 suppression of mitogenesis was reversed by ACI (Hoegy et al., 2001). The timing of the increase in PKA coincided with a significant decrease in the cellular proliferative potential (Liu et al., 1986).

Senescence-associated cAMP accumulation by LPA results in the activation of PKA (fang et al., 2006b), which would reduce DNA synthesis through the Raf/ERK signal transduction (Chuang et al., 1994; Mischak et al., 1996; Wu et al., 1993). cAMP levels can be readily modulated by many metabolic and hormonal stimuli. The role of cAMP in the regulation of mitogenic responses in senescent cells suggests that modulation of cAMP can be a potential tool for influencing the aging process and its associated degenerative changes. From these observations, the inventors conjecture that the reported effects of calorie restriction on life span extension and many ageing-related physiological and pathological changes, would need to be reassessed in terms of cAMP status as well (the role of cAMP is important as such).

LPA can induce cell migration by activating Rho through the heterotrimeric GTPases G₁₂/G₁₃ in neuronal cells (Kranenburg et al., 1999), and G₁₃ in Swiss 3T3 cells (Gohla et al., 1998) and fibroblast cell lines derived from wild-type and Gαq/Gβ11-deficient mice. Our results show that LPA enhances cell migration significantly both in young and senescent cells (FIG. 6). Agents that increase fibroblast cAMP levels, including dbcAMP, forskolin, PGE, and isoproterenol, can inhibit fibroblast migration by activation of PKA (Kohyama et al., 2001). The cAMP/PKA signal transduction has also been shown to inhibit smooth muscle cell (SMC) migration (Goncharova et al., 2003; Horio et al., 1995; Itoh et al., 2001; Sun et al., 2002; Zhu and Hui, 2003). The inhibitory effect of cAMP may be mediated by inhibition of RhoA (Buchan et al., 2002; Chen et al., 2005; Mukai et al., 2000). In young fibroblasts, LPA might activate Rho fully by inhibition of PKA, while in senescent cells, Rho activates a portion of PKA activity, causing reduced migration of senescent cells (Jang et al., 2006b). Thus, modulation of cAMP by dbcAMP or ACI could alter the rate of senescent cell migration. In the present invention, it is confirmed that LPA-induced cell migration is reduced by dbcAMP (by reducing cAMP levels). As the inventors expected, senescent cell migration was enhanced by co-treatment with both ACI and LPA more significantly than by LPA treatment alone (FIG. 6). When ACI was applied with or without LPA to the wounded rats, cell proliferation is increased significantly (FIG. 7-9). The effects of LPA or ACI were more evident in aged rats than in the young. This suggests that co-treatment with ACI and LPA may be a better choice for the cure of skin wounds of aged animals.

Various cAMP-elevating agents, including dbcAMP, forskolin, cholera toxin, and 3-isobutyryl-1-methylxanthine, consistently inhibit LPA-induced cell migration (in vitro invasion) of rat ascites hepatoma cells (Mukai et al., 2000). Alpha6beta4 integrin suppresses the intracellular cAMP concentration and is a major cause of chemotactic migration in MDA-MB-435 breast carcinoma cells (O'Connor et al., 1998).

The crosstalk between cAMP/PKA and RhoA-mediated signal transductions has also significant affect on biologic features, such as morphologic and cytoskeletal changes, migration, and anchorage-independent growth of gastric and prostate cancer cells (Chen et al., 2005).

In summary, the invention indicates that reduction in cAMP levels is a major factor that modulates proliferation and migration of senescent, and skin wound healing in aged animals. This finding has the potential to be extended to wound healing for aged humans.

### REFERENCES

Agren, M. S., Steenfos, H. H., Dabelsteen, S., Hansen, J. B., Dabelsteen, E., 1999. Proliferation and mitogenic response to PDGF-BB of fibroblasts isolated from chronic venous leg ulcers is ulcer-age dependent. J. Invest. Dermatol. 112, 463-469.
An, S., Bleu, T., Hallmark, O. G., Goetzl, E. J., 1998. Characterization of a novel subtype of human G protein-coupled receptor for lysophosphatidic acid. J. Biol. Chem. 273, 7906-7910.
Balazs, L., Okolicany, J., Ferrebee, M., Tolley, B., Tigyi, G., 2001. Topical application of the phospholipid growth factor lysophosphatidic acid promotes wound healing in vivo. Am. J. Physiol. Regul. Integr. Comp. Physiol. 280, R466-472.
Bandoh, K., Aoki, J., Hosono, H., Kobayashi, S., Kobayashi, T., Murakami-Murofushi, K., Tsujimoto, M., Arai, H., Inoue, K., 1999. Molecular cloning and characterization of a novel human G-protein-coupled receptor, EDG7, for lysophosphatidic acid. J. Biol. Chem. 274, 27776-27785.
Bryant, R., 2007. Acute and Chronic Wounds: Current Management Concepts. 3^{rd} ed. Philadelphia: Mosby, Elsevier.
Boyce, S. T., Ham, R. G., 1983. Calcium-regulated differentiation of normal human epidermal keratinocytes in chemically defined clonal culture and serum-free serial culture. J. Invest. Dermatol. 81, 33s-40s.
Buchan, A. M., Lin, C. Y., Choi, J., Barber, D. L., 2002. Somatostatin, acting at receptor subtype 1, inhibits Rho activity, the assembly of actin stress fibers, and cell migration. J. Biol. Chem. 277, 28431-28438.
Chen, Y., Wang, Y., Yu, H., Wang, F., Xu, W., 2005. The cross talk between protein kinase A- and RhoA-mediated signaling in cancer cells. Exp. Biol. Med. (Maywood) 230, 731-741.
Cho, K. A., Ryu, S. J., Park, J. S., Jang, I. S., Ahn, J. S., Kim, K. T., Park, S. C., 2003. Senescent Phenotype Can Be Reversed by Reduction of Caveolin Status. J. Biol. Chem. 278, 27789-27795.
Chuang, E., Barnard, D., Hettich, L., Zhang, X. F., Avruch, J., Marshall, M. S., 1994. Critical binding and regulatory interactions between Ras and Raf occur through a small, stable N-terminal domain of Raf and specific Ras effector residues. Mol. Cell. Biol. 14, 5318-5325.
Contos, J. J., Ishii, I., Chun, J., 2000. Lysophosphatidic acid receptors. Mol. Pharmacol. 58, 1188-1196.
Erickson, J. R., Espinal, G., Mills, G. B., 2000. Analysis of the EDG2 receptor based on the structure/activity relationship of LPA. Ann. N.Y. Acad. Sci. 905, 279-281.
Fabbri, E., Brighenti, L., Ottolenghi, C., 1991. Inhibition of adenylate cyclase of catfish and rat hepatocyte membranes by 9-(tetrahydro-2-furyl)adenine (SQ 22536). J. Enzyme. Inhib. 5, 87-98.
Fukushima, N., Chun, J., 2001. The LPA receptors. Prostaglandins. Other. Lipid. Mediat. 64, 21-32.
Fukushima, N., Ishii, I., Contos, J. J., Weiner, J. A., Chun, J., 2001. Lysophospholipid receptors. Annu. Rev. Pharmacol. Toxicol. 41, 507-534.
Goetzl, E. J., An, S., 1998. Diversity of cellular receptors and functions for the lysophospholipid growth factors lysophosphatidic acid and sphingosine 1-phosphate. Faseb. J. 12, 1589-1598.
Gohla, A., Harhammer, R., Schultz, G., 1998. The G-protein G13 but not G12 mediates signaling from lysophosphatidic acid receptor via epidermal growth factor receptor to Rho. J. Biol. Chem. 273, 4653-4659.
Gohla, A., Offermanns, S., Wilkie, T. M., Schultz, G., 1999. Differential involvement of Galpha12 and Galpha13 in receptor-mediated stress fiber formation. J. Biol. Chem. 274, 17901-17907.
Goncharova, E. A., Billington, C. K., Irani, C., Vorotnikov, A. V., Tkachuk, V. A., Penn, R. B., Krymskaya, V. P., Panettieri, R. A., Jr., 2003. Cyclic AMP-mobilizing agents and glucocorticoids modulate human smooth muscle cell migration. Am. J. Respir. Cell. Mol. Biol. 29, 19-27.
Hardman, M., Ashcroft, G., 2008. Estrogen, not intrinsic aging, is the major regulator of delayed human wound healing in the elderly. Genome. Biology. 9, R80.
Hoegy, S. E., Oh, H. R., Corcoran, M. L., Stetler-Stevenson, W. G., 2001. Tissue inhibitor of metalloproteinases-2 (TIMP-2) suppresses TKR-growth factor signaling independent of metalloproteinase inhibition. J. Biol. Chem. 276, 3203-3214.
Horio, T., Kohno, M., Kano, H., Ikeda, M., Yasunari, K., Yokokawa, K., Minami, M., Takeda, T., 1995.

Adrenomedullin as a novel antimigration factor of vascular smooth muscle cells. Circ. Res. 77, 660-664.
Itoh, H., Yamamura, S., Ware, J. A., Zhuang, S., Mii, S., Liu, B., Kent, K. C., 2001. Differential effects of protein kinase C on human vascular smooth muscle cell proliferation and migration. Am J Physiol, Heart, Circ, Physiol, 281, H359-370.
Jahovic, N., Guzel, E., Arbak, S., Yegen, B. C., 2004. The healing-promoting effect of saliva on skin burn is mediated by epidermal growth factor (EGF): role of the neutrophils. Burns. 30, 531-538.
Jang, I. S., Juhnn, Y. S., 2001. Adaptation of cAMP signaling system in SH-SY5Y neuroblastoma cells following expression of a constitutively active stimulatory G protein alpha, Q227L Gsalpha. Exp. Mol. Med. 33, 37-45.
Jang, I. S., Rhim, J. H., Kim, K. T., Cho, K. A., Yeo, E. J., Park, S. C., 2006a. Lysophosphatidic acid-induced changes in cAMP profiles in young and senescent human fibroblasts as a clue to the ageing process. Mech. Ageing. Dev. 127, 481-489.
Jang, I. S., Rhim, J. H., Park, S. C., Yeo, E. J., 2006b. Downstream molecular events in the altered profiles of lysophosphatidic acid-induced cAMP in senescent human diploid fibroblasts. Exp. Mol. Med. 38, 134-143.
Jang, I. S., Yeo, E. J., Park, J. A., Ahn, J. S., Park, J. S., Cho, K. A., Juhnn, Y. S., Park, S. C., 2003. Altered cAMP signaling induced by lysophosphatidic acid in senescent human diploid fibroblasts. Biochem. Biophys. Res. Commun. 302, 778-784.
Kohyama, T., Ertl, R. F., Valenti, V., Spurzem, J., Kawamoto, M., Nakamura, Y., Veys, T., Allegra, L., Romberger, D., Rennard, S. I., 2001. Prostaglandin E(2) inhibits fibroblast chemotaxis. Am. J. Physiol. Lung. Cell. Mol. Physiol. 0.281, L1257-1263.
Kranenburg, O., Poland, M., van Horck, F. P., Drechsel, D., Hall, A., Moolenaar, W. H., 1999. Activation of RhoA by lysophosphatidic acid and Galpha12/13 subunits in neuronal cells: induction of neurite retraction. Mol. Biol. Cell. 10, 1851-1857.
Liu, A. Y., Chang, Z. F., Chen, K. Y., 1986. Increased level of cAMP-dependent protein kinase in aging human lung fibroblasts. J. Cell. Physiol. 128, 149-154.
Marcus, J. R., Tyrone, J. W., Bonomo, S., Xia, Y., Mustoe, T. A., 2000. Cellular mechanisms for diminished scarring with aging. Plast. Reconstr. Surg. 105, 1591-1599.
Mischak, H., Seitz, T., Janosch, P., Eulitz, M., Steen, H., Schellerer, M., Philipp, A., Kolch, W., 1996. Negative regulation of Raf-1 by phosphorylation of serine 621. Mol. Cell. Biol. 16, 5409-5418.
Moolenaar, W. H., 2000. Development of our current understanding of bioactive lysophospholipids. Ann. N.Y. Acad. Sci. 905, 1-10.
Mukai, M., Nakamura, H., Tatsuta, M., Iwasaki, T., Togawa, A., Imamura, F., Akedo, H., 2000. Hepatoma cell migration through a mesothelial cell monolayer is inhibited by cyclic AMP-elevating agents via a Rho-dependent pathway. FEBS Lett. 484, 69-73.
Noguchi, K., Ishii, S., Shimizu, T., 2003. Identification of p2y9/GPR23 as a novel G protein-coupled receptor for lysophosphatidic acid, structurally distant from the Edg family. J. Biol. Chem. 278, 25600-25606.
O'Connor, K. L., Shaw, L. M., Mercurio, A. M., 1998. Release of cAMP gating by the alpha6beta4 integrin stimulates lamellae formation and the chemotactic migration of invasive carcinoma cells. J. Cell. Biol. 143, 1749-1760.
Pages, C., Simon, M. F., Valet, P., Saulnier-Blache, J. S., 2001. Lysophosphatidic acid synthesis and release. Prostaglandins Other Lipid Mediat. 64 1-10.
Park, W. Y., Park, J. S., Cho, K. A., Kim, D. I., Ko, Y. G., Seo, J. S., Park, S. C., 2000. Up-regulation of Caveolin Attenuates Epidermal Growth Factor Signaling in Senescent Cells. J. Biol. Chem. 275, 20847-20852.
Piazza, G. A., Ritter, J. L., Baracka, C. A., 1995. Lysophosphatidic acid induction of transforming growth factors alpha and beta: modulation of proliferation and differentiation in cultured human keratinocytes and mouse skin. Exp. Cell. Res. 216, 51-64.
Pittman, J., 2007. Effect of aging on wound healing: current concepts. J. Wound. Ostomy. Continence. Nurs. 34, 412-415.
Rhim, J. H., Jang, I. S., Song, K. Y., Ha, M. K., Cho, S. C., Yeo, E. J., Park, S. C., 2008. Lysophosphatidic Acid and adenylyl cyclase inhibitor increase proliferation of senescent human diploid fibroblasts by inhibiting adenosine monophosphate-activated protein kinase. Rejuvenation Res. 11, 781-792
Seternes, O. M., Sorensen, R., Johansen, B., Moens, U., 1999. Activation of protein kinase A by dibutyryl cAMP treatment of NIH 3T3 cells inhibits proliferation but fails to induce Ser-133 phosphorylation and transcriptional activation of CREB. Cell Signal. 11, 211-219.
Shiraha, H., Glading, A., Chou, J., Jia, Z., Wells, A., 2002. Activation of m-calpain (calpain II) by epidermal growth factor is limited by protein kinase A phosphorylation of m-calpain. Mol. Cell. Biol. 22, 2716-2727.
Shiraha, H., Gupta, K., Drabik, K., Wells, A., 2000. Aging fibroblasts present reduced epidermal growth factor (EGF) responsiveness due to preferential loss of EGF receptors. J. Biol. Chem. 275, 19343-19351.
Spindler, K. P., Nanney, L. B., Davidson, J. M., 1995. Proliferative responses to platelet-derived growth factor in young and old rat patellar tendon. Connect. Tissue Res. 31, 171-177.
Sun, J., Sui, X., Bradbury, J. A., Zeldin, D. C., Conte, M. S., Liao, J. K., 2002. Inhibition of vascular smooth muscle cell migration by cytochrome p450 epoxygenase-derived eicosanoids. Circ. Res. 90, 1020-1027.
Takuwa, Y., Takuwa, N., Sugimoto, N., 2002. The Edg family G protein-coupled receptors for lysophospholipids: their signaling properties and biological activities. J. Biochem (Tokyo). 131, 767-771.
Wang, H. J., Wan, H. L., Yang, T. S., Wang, D. S., Chen, T. M., Chang, D. M., 1996. Acceleration of skin graft healing by growth factors. Burns. 22, 10-14.
Whang, W. K., Park, H. S., Ham, I. H., Oh, M., Namkoong, H., Kim, H. K., Hwang, D. W., Hur, S. Y., Kim, T. E., Park, Y. G., Kim, J. R., Kim, J. W., 2005. Methyl gallate and chemicals structurally related to methyl gallate protect human umbilical vein endothelial cells from oxidative stress. Exp. Mol. Med. 37, 343-352.
Wu, J., Dent, P., Jelinek, T., Wolfman, A., Weber, M. J., Sturgill, T. W., 1993. Inhibition of the EGF-activated MAP kinase signaling pathway by adenosine 3′,5′-monophosphate. Science. 262, 1065-1069.
Yeo, E. J., Hwang, Y. C., Kang, C. M., Choy, H. E., Park, S. C., 2000. Reduction of UV-induced cell death in the human senescent fibroblasts. Mol. Cells. 10, 415-422.
Yeo, E. J., Jang, I. S., Lim, H. K., Ha, K. S., Park, S. C., 2002. Agonist-specific differential changes of cellular signal transduction pathways in senescent human diploid fibroblasts. Exp. Gerontol. 37, 871-883.
Zhu, Y., Hui, D. Y., 2003. Apolipoprotein E binding to low density lipoprotein receptor-related protein-1 inhibits cell migration via activation of cAMP-dependent protein kinase A. J. Biol. Chem. 278, 36257-36263.

1. A pharmaceutical composition for promoting wound healing, comprising lysophosphatidic acid and adenylyl cyclase inhibitor as active ingredients.

2. The pharmaceutical composition according to claim 1, wherein the adenylyl cyclase inhibitor is SQ22536.

3. The pharmaceutical composition according to claim 1, wherein a target subject for wound healing is an aged mammal.

4. The pharmaceutical composition according to claim 3, wherein the mammal is human.

5. The pharmaceutical composition according to claim 1, wherein the composition is topically applied to the wound.

6. The pharmaceutical composition according to claim 1, wherein the composition is orally administered.

7. The pharmaceutical composition according to claim 1, wherein the composition is parenterally administered.

8. A method for promoting wound healing, comprising treating a target subject with an effective amount of lysophosphatidic acid and adenylyl cyclase inhibitor.

9. The method for promoting wound healing according to claim 8, wherein the target subject for wound healing is an aged mammal.
